(19) United States
(12) Patent Application Publication
(10) Pub. No.: US 2010/0152554 A1
(43) Pub. Date: Jun. 17, 2010

(54) MONITORING DEVICE

(76) Inventors: Matthias Steine, Munich (DE); Eurig Wyn Jones, Munchen (DE)

Correspondence Address: Fanelli Strain & Haag PLLC, 1455 Pennsylvania Ave., N.W., suite 400, Washington, DC, 20004, US

(21) Appl. No.: 12/518,924

(22) PCT Filed: Dec. 14, 2007
(86) PCT No.: PCT/EP2007/011026

§ 371 Date: Jan. 19, 2010

Related U.S. Application Data
(63) Continuation-in-part of application No. PCT/EP2006/012080, filed on Dec. 14, 2006.

(51) Int. Cl. A61B 5/145

(52) U.S. Cl. 600/309

(57) ABSTRACT

Provided is a small sized, portable monitoring device capable of determining an analyte under investigation and having a system and method for providing compliance information to a user of his management of a disease, and for easily navigating a menu structure by means of manual control(s). Further provided is the provision of feedback to the user in form of a disease management information to be easily understood by a user such as the further described COMPLIANCE WINDOW or the INDICATOR CATEGORIES. The user interface can be used in connection with a glucose diagnostic device, a coagulation diagnostic device, immunoassay diagnostic device, and other monitoring devices such as an blood pressure monitor or a pedometer.

### FIELD OF THE INVENTION

The invention relates to the field of physiological monitoring and diagnostic devices and more particularly, to the provision of information for use in connection with personal monitoring devices.

### BACKGROUND OF THE INVENTION

The use of monitoring devices e.g. meters, at the point of care has become increasingly common and prevalent over the last few years with the development of electronic miniaturisation techniques, improved test element technology, and the increasing number of individuals eager to self-manage their diseases.

For instance, people suffering from chronic diseases such as diabetes mellitus and/or blood clotting problems have, under the orders of a health care professional, to regularly undertake a test to aid in the management of their disease. Typically, a test element is used in combination with a monitoring device to determine the presence and concentration of an analyte in a sample of physiological fluid utilising one of several detection principles (e.g. electrochemical, photometric to name but a few). The user may then be informed, typically by means of a displayed numerical value, of the concentration of an analyte under investigation.

Common to all devices is the provision of a user interface. Typically, the user interface includes an input console as well as a display screen on which a menu and test results are displayed. The input console may include a number of manual controls such as switches and/or jog-wheels that may allow a user to manipulate the information displayed on the display screen, e.g. for operating the device, for viewing results, and optionally for configuring a health management plan.

Indeed easy navigation of a user interface is of paramount consideration when designing such personal diagnostic devices and some consideration is therefore given to their utilisation, for providing accessibility to as broad a demographic range as possible. For instance, designers of devices earmarked for the diabetes market face several challenges, not least due to the elevated risk of cerebrovascular accidents (e.g. strokes) seen with some diabetic patients as a consequence of poor glycemic control. However, personal diagnostic devices are characterised by small display screens, limited input capability, and a numeric representation of the concentration of an analyte in a sample of physiological fluid. Accordingly the operation and displacement of manual controls of an input console may therefore prove to be difficult to all but healthy individuals, potentially leading to non-compliance of a recommended treatment regimen.

User navigation of a menu displayed on the display screen may be possible but the input of data believed to be important for good glycemic control (e.g. data relating to dietary habits, exercise activity, medication information and so on) into the device may prove to be challenging and difficult.

Whilst personal diagnostic devices have become more sophisticated over recent times by virtue of improved technology, an increased number of functions have subsequently been added. Generally however, as more functions are added, additional manual controls are added, causing more device operability complications to individuals especially those that have dexterity problems and/or those that are poor sighted. Clearly, since personal diagnostic devices are considered portable there is a practical limit on a useful device size.

Nevertheless, personal diagnostic devices rarely vary in capability (screen sizes, data output, supported technologies etc.) and a “one size fits all” design methodology is however a common strategy by device designers. In addition, standardisation of capability tends to approach a lowest common denominator (e.g. provision of basic measurement result).

Furthermore health practitioners currently recommend that diabetic patients self monitor their glucose concentration values several times per day. It is generally known however that the numerical values generated i.e. glucose measurements resulting from a test, will vary during the day depending on several factors (e.g. dietary habits, exercise regime, medication and so on). Important therefore for the patient is the need to comprehend the resulting measurement values and the impact and influence of the aforementioned factors has on the patient's condition. For instance, whilst several measurement values may be generated over the day, it is currently cumbersome for an individual to understand the importance of the data that is generated, thus insufficient information maybe gleaned from a display screen of a diagnostic device by the patient to fully understand the impact his lifestyle may have on his measurement values. Additionally, whilst known diagnostic devices provide an averaging or mean function, a function which is an important parameter in aiding a user during management of a disease, it does however have limitations in the information that is presented to the user. For example, there is a possibility that the user may experience dangerous high or low glucose concentrations, so a mean value presented over a predetermined period of time (e.g. 7 or 14 days) might be considered misleading. Furthermore, such averaging functions are considered impractical to use since they do not relate to user specific events or highlight deviations from target measurements. Similarly, some users may try to manipulate their mean concentration measurement values by systematically repeating good measurements such that the mean values are improved.

Accordingly, since the measurement values may be variable upon dependence of dietary habits, exercise regime, medication and so on, questioning the influences of these factors is essential for validly interpreting the measurement values. There is, therefore a desire to enable a user to be informed of deviations from target measurement values to allow a user to easily make informed decisions for subsequent changes to his lifestyle.

One common approach in attempting a user to comprehend the measurement values generated by diagnostic devices is the provision of data download tools such that a diagnostic device is connected by wire or wireless methods to a personal computer running specialist software. However, such downloading of data is a time consuming task and relies on the user being in possession of a personal computer. Furthermore, the user has to learn how to manipulate such specialist software thus undermining the effectiveness and instantaneity of understanding the influences and factors affecting the variability of measurement values.

A further approach in reducing some of the drawbacks associated with interfacing a device to a user is to provide physiological data to a user by auditory means such as a speaker.

An interesting prior art publication to address the problem interfacing a glucose measuring device to a user is that described in United States Patent Application 2004/0015102 and published to Cummings et al., on 22 Jan. 2004. However, manipulation of the user interface of such a glucose measuring device relies solely on the depression of the manual controls.

Web publication http://www.agamatrix.com/product_wave_{—}1.shtml discloses a glucose diagnostic device incorporating a bar graph function for comparing average readings over predetermined time frames. However, the resulting analysis only provides a trend of average readings.

WO2005001680 published 6^{th} January to Hansen, discloses a user interface for a portable medical device, including a display screen and virtual switches, with a user input device allowing the virtual switches of the GUI to be selected. Similarly, the manipulation and navigation of the user interface of such a medical device depends on the user being able to use a pointing device;

WO2005009205 published 3 Feb. 2005 to Anderson et al., discloses a system and method for self management of health using natural language interface, initiating dialogue with a user to solicit health information from the user using a natural language interface and may respond unsolicited health information from the user provided via the natural language interface. The health information from the user is semantically processed in accordance with pre-specified health management rules to facilitate user self management of health. The natural language interface is a constrained natural language. However, such a system requires connection to a cell phone for instance.

Sensory Inc, Data sheet in relation to RSG-4128 microprocessor

In view of the aforementioned prior art publications, it is an object of the present invention to provide a system and method of a personal diagnostic device which provides an interface to a user for providing an analyte result not only in numerical form but also in graphical and/or iconic form. It a further object of the present invention to provide an indication to a user whether he is effectively complying with his treatment regimen. It is yet another object of the present invention to provide an easy and intuitive navigation of a menu system and sub-menu system of the personal diagnostic device. It is also desirable that the device includes a data interface, thus permitting a user to connect the device to a network or personal computer. There is also a need for such a device which is low cost and can be easily implemented using readily available components.

The invention aims to alleviate at least some of the above identified problems and/or needs.

### SUMMARY OF THE INVENTION

A monitoring device according to embodiments of the present invention comprises a processor, and a built-in sensor or a port equipped with a disposable sensor. The monitoring device is adapted for relating the stored measurement values of the sensor to a medical useful compliance range. By relating the measurement value to a predefined compliance range, the user may immediately recognize how an actual measurement value determined by the sensor is located relative to a predefined compliance range of medical useful values. Thus, the user may judge how good or bad the actual measurement value is.

In a preferred embodiment, evaluation of measurement values is performed with regard to a predefined set of specific events that occur periodically during a period of 24 hours, i.e. during a period of time comprising one day and one night. The specific events may e.g. comprise meal type events. These meal type events comprise pre-prandial and post-prandial events such as e.g. pre-breakfast, post-breakfast, pre-lunch, post-lunch, pre-dinner, post-dinner, etc. . . . Furthermore, the specific events may e.g. comprise events related to medication

According to a preferred embodiment, an event is defined by a characteristic time frame, e.g. the event “post-breakfast” might range from 9 AM to 11 AM. In a further preferred embodiment, analysis of the measurement values is performed in accordance with the respective event. For example, a mean value of measurement values acquired during the last few days might be determined, whereby only those stored measurement values with a time stamp that lies within the time frame related to the respective event are considered. Thus, for each of the time frames related to a specific event, a separate mean value may e.g. be determined.

According to a preferred embodiment, the set of specific events and the related time frames together form a compliance management profile that reoccurs every 24 hours. In this embodiment, a current measurement value may be compared with the statistical properties of former measurement values that belong to the same event, like e.g. pre-lunch. For example, a current measurement value that has been acquired in the time frame “pre-lunch” may be compared with a mean value of the “pre-lunch” measurements acquired during the past days. By analysing measurement values in accordance with the time frame they belong to, a set of mean values and statistical properties is built up automatically. This “data base” of former values is the base point for analysis of the current measurement.

The advantage of using compliance management profiles comprising a set of predefined specific events is that the dependence of the measurement values on the respective time of day can be considered. A user may compare an actual measurement value both with a compliance window and with a mean value of the time frame the actual measurement value belongs to. Thus, the position of the actual measurement value relative to the mean value of the past few days can be shown to the user. This provides for a more thorough understanding of the measurement values, which are perceived together with a context of former values.

### BRIEF DESCRIPTION OF DRAWINGS

A better understanding of the features and advantages of the present invention will be obtained by reference to the following detailed description that sets forth illustrative embodiments by way of example only, in which the principles of the invention are utilized, and the accompanying drawings of which:

FIG. 1 shows a simplified block diagram of a first main element of the diagnostic device according to an embodiment of the present invention;

FIG. 2 shows a simplified block diagram of the first main element in electrical connection to a second main element according to an embodiment of the present invention;

FIG. 3 shows a display screen with an example screen layout providing a numerical result area, several information icons, and an information section according to an embodiment of the present invention;

FIG. 4 shows a display screen with an example screen layout, providing a numerical result area, several information icons, and an information section depicting a disease management compliance window;

FIG. 5 shows an enlarged view of the information section of FIG. 4 showing a disease management compliance window in greater detail;

FIG. 6 shows an enlarged view of the information section of FIG. 3 showing a disease management compliance alphanumerical message;

FIG. 7 shows a display screen with an example screen layout providing a numerical result area, several information icons and several disease compliance indicators, according to another embodiment of the present invention;

FIG. 8a shows an enlarged view of the information section of FIG. 3 showing an interactive user navigational area of an interface program according to an embodiment of the present invention;

FIG. 8b shows a first level menu of an interface program of a personal diagnostic device according to the embodiment of FIG. 8a;

FIG. 8c shows a second level menu of an interface program of a personal diagnostic device according to the embodiment of FIG. 8a.

FIG. 9a shows a flow chart showing the steps of meal marking in relation to a measurement sequence.

FIG. 9b shows a schematic view of a user selectable menu for a pre or post prandial meal event.

FIG. 9c shows a display screen showing a result screen populated with a meal category and a graphical tool.

FIG. 9d shows a display screen showing a result screen populated with a meal category.

### DETAILED DESCRIPTION OF THE INVENTION

For specific chronic diseases or conditions a strict therapeutic treatment routine is mandatory to achieve a satisfying compliance, thus avoiding long-term complications or toxic reactions due to incorrect dosed drugs. The most common examples for these types of conditions are diabetes or coagulation disorders. In the case of diabetes, the diagnostic results should be related to the indicative events such as meals and the administration of insulin, because both events will have a strong impact on the blood glucose concentration. Furthermore, it is typical for diabetes that patients follow a predetermined schedule balancing food in-take, exercise, and the administration of a mixture of short acting and long acting insulin for their personal therapy. One skilled in the art will agree that the applied amount of insulin will effect subsequent blood glucose measurements. The disclosed invention provides a useful tool for a user to follow a predetermined schedule with a predetermined set of events and relating this schedule to the acquired diagnostic measurement data. Thus, the patient will keep always an overview over certain behavioural manners connected to a certain event i.e. if the “style of eating” at dinner time may cause a satisfying or non satisfying compliance. The disclosed invention in form of the monitoring device provides the possibility to calculate average and/or statistical representations of measurement values according to a “compliance management profiles”, which represents a plurality of periodically reoccurring special events such as a mealtime i.e. breakfast, lunch, dinner, or other events which influences the “life style” of a person i.e. wake-up time or bed-time. All these events in terms of the activity such as food in-take, exercise or administration of medicine are of central importance for the compliance of the patient.

With aid of the compliance management profiles the personal monitoring device has the means to single out certain measurement values and provides due to the connection of this data point to a specific event a highly improved interpretation of the diagnostic result for the user or the physician. One skilled in the art will agree that this type of monitoring device has a great advantage over a manually kept diary, which is currently the common praxis for many (diabetes) patients.

The utility of the compliance management profile can be appreciated easily if its function is understood as an automatic filter or sorting algorithm, which processes certain measurements values due to a predetermined time scheme (understood and described below as management time frame), whereas such functionality is activated or deactivated by the real time clock (RTC) of a host processor. With other words, the discussed algorithm would label the measurement value with a specific event label in this case a meal type identifier such as “Breakfast” or “Dinner” depending on the preset of the compliance management profile and the RTC of a host processor.

A further aspect of the invention is the provision of a self-learning mode of the disclosed monitoring device. As such the user can label a measurement value specifically as “Breakfast”, “Dinner”, “Exercise”, or “Administration of insulin” and optionally with one of the additional event labels “Pre” or “Post” to generate for example the event “Pre-Breakfast” to indicate a measurement value before breakfast. In case the user labels a certain measurement value manually using the analogue input device of the monitoring device, a host processor is configured in a way that the compliance management profile is automatically updated and the monitoring device will learn the daily routine of the patient or the device user, respectively.

Compliance Management Profile—Example 1

For the sake of discussing the compliance management profile we assume a hypothetical diabetes patient injecting insulin shortly before bedtime. In the first case the patient injects an insulin dose below his requirements leading to high blood glucose measurement value before breakfast. In the second case the patient injects an insulin dose above his requirements leading to a dangerous hypo-glycaemia before breakfast. As easily understood it would be of importance for the patient to know if a) the actual glucose concentration before breakfast is correct, to low, or to high and b) if that measurement result is a “one off” or if all measurements of the specific “Pre-Breakfast” event of i.e. the last 7 days are correct, to low, or to high. The correct information at this point can help to adapt the treatment regime if necessary.

Furthermore, it becomes obvious that in the given cases a simple 7 day average method may hide and/or even out fluctuations of the blood glucose values occurring at specific events i.e. meal types.

In summary the compliance management profile can be understood as a instruction set for a host processor to link certain measurement values to specific and/or reoccurring events as described above, whereas the disease management compliance window or simply the compliance window can be understood as “look-up indicator” allowing the user a brief orientation of his current compliance compared to the long-term compliance i.e. over the last 7, 14 or 30 days at specific events such as “Pre-Breakfast” or alternatively for the entire day.

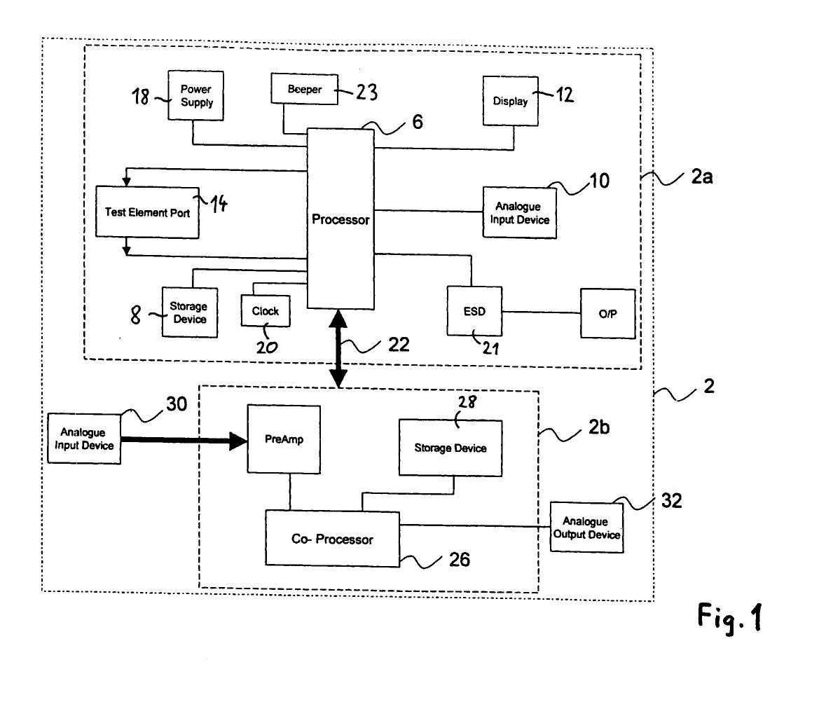

Now referring to FIG. 1 a simplified block diagram of a system 2 according to an embodiment of the present invention is shown. Block diagram depicts two main elements 2a, 2b, electrically connected to each other. The two main elements 2a, 2b may conveniently be housed in a housing (not shown) of suitable size and shape to be grasped by a user's hand, and made from a polymeric material.

The first main element 2a shows a microprocessor 6 electrically connected to a storage device 8 such as a ROM and RAM, an analogue input device 10, a display screen 12, an analyte test element port 14, an output connector, a power supply 18, an oscillator circuit 20 and a bus 22. The microprocessor 6 of the first main element 2a will be regarded, according to the present invention, as a host processor 6. Preferably, the microprocessor is a 16 bit microprocessor such as the MAXQ2000 microprocessor available from Dallas Semiconductor Corporation, 4401 South Beltwood Parkway, Dallas, Tex., USA. It will be appreciated by those of ordinary skill in the art that while a MAXQ2000 microprocessor or faster microprocessor is preferred, any other microprocessor, either available presently or in the future, could be utilised.

Generally, host processor 6 has several input and output ports operating in conjunction with additional circuits such as analogue to digital converters, storage devices (e.g. RAM and ROM), and the oscillator circuit 20 for maintaining correct timing of digital signals. Additionally a low power circuit configuration maybe provided as part of host processor 6, for allowing the system 2 to conserve battery power during idle moments. Such a circuit generally controls analogue circuits thereby shutting them off when not in use.

Indeed, host processor 6 additionally has the capability for storing of operational software that controls standard operation of the diagnostic device, for storing software to operate a user interface, and for storing several algorithms which evaluate several input signals (e.g. from sensors and/or interfaced signals) as will be described in greater detail below. Examples of such algorithms include statistical analysis algorithms, data analysis algorithms and so on. Host processor 6 may additionally store test measurement data in the form of analyte test result records, or such data may be stored on the aforementioned external storage devices 8. Such external storage devices can include static or dynamic RAM, non-static RAM, rewritable ROMs, flash memory, or the like. Electrical Static Discharge protection may be provided by an integrated circuit 21 such as those provided by Dallas Semiconductor Corporation such that uncontrolled microprocessor operation is minimized.

Further electrically connected to the host processor 6 is the analyte test element port 14. The port 14 can take various forms and may include electrical connectors, a microswitch, and/or sensors (not shown) for cooperating with an inserted test element. In one configuration, the electrical connectors of the test element port 14 may correspond to electrical contacts of an electrochemical test element for example. Preferably however, the port 14 houses an optical system. Such an optical system includes but not limited to, at least one light source (e.g. LED) and at least one optical sensor (e.g. photodiode), and configured such that the optical system corresponds to at least one reaction area of an optical test element.

The analysis algorithms, stored on the host processor, are controlled by the operational software and in one aspect of the present invention determine the concentration of an analyte under investigation utilizing signals received from the analyte test element port. For example, the data analysis algorithm(s) may be configured for ensuring that a glucose concentration may be correctly calculated using a test element of European Patent Specification 1574858 the contents of which are disclosed herein by reference. Similarly, embedded data analysis algorithm may be adapted for calculating the coagulation time of a sample of physiological fluid (e.g. blood) using test element of Co-Pending Patent Application PCT/EP2005/009382 and herein incorporated by reference, alternatively embedded data analysis algorithm may be adapted to cooperate with an immunoassay test sensor of Co-Pending Patent Application PCT/EP2005/009381 and herein incorporated by reference. The signals are received on an input/output (I/O) port of the host processor 6 after processing by sub-circuits such as converter circuitry, filter circuitry and/or amplifiers.

Further depicted in the current illustration, is the analogue input device 10. The analogue input device 10, forming part of the user interface, may encompass manual switches (not shown). As will be briefly described later, analogue input device may take other forms 30. The manual switches may be constituted by scroll switches, a rotary wheel, and/or a confirmation switch. Additionally, alphanumerical or non-alphanumerical characters (not shown) may form part of the manual switches for aiding the user in correct displacement thereof. Optionally, the manual switches may be backlit by means of LED's for further aiding the user in the displacement of the switches, especially in low light conditions.

Further shown in the block diagram of FIG. 1 is a display screen 12. The display screen 12, may be implemented by a commonly sourced colour or monochromatic dot matrix type, and/or segmented type display, and/or hybrid segmented-dot matrix type, and/or organic LED type display screen, or any other display screen available presently or in the future, and connected to an output port of the host processor. Driving circuit for the display screen 12 is provided by the host processor 6 such that only minimal additional components are required to realise the functionality of the display screen 12. The display screen 12 may however include its own driving circuitry. The display screen 12 forms part of the user interface 4 and as such provides information to the user for navigating the operational menus of an interface program and for providing a means of reporting information to a user (e.g. information relating to an analyte under investigation).

Analyte test result data may optionally be downloaded to a peripheral device (e.g. Personal Computer) for further processing and analysis by utilizing the output connector of the system. For instance, output connector connected to the host processor 6 allows connectivity and data download by wire to a personal computer, mobile telephone and so on. Such downloaded data may be manipulated by a user using commercially available data management software, widely known in the art with no further details required herein.

Optionally, a beeper 23 such as a piezoelectric transducer may be provided and connected by means of an operational amplifier circuit to the host processor 6 as would be known to persons skilled in the art. Beeper 23 is for providing a response to a user for alerting of various functional features (e.g. meal time, alarm, incorrect analogue input device usage and so on of the device). User may control whether to utilize such a response by means of interface program as will be described later.

The block diagram also includes a power supply 18 such as at least one commonly sourced battery. Such a battery may be a Lithium CR2032 type battery, an AA type battery or an AAA type battery. A low battery alarm (such as display of a graphical symbol on the display screen and/or driving of the beeper) may be included with the main operating program with a threshold trigger optionally configured by a user. Optionally, beeper 23 may provide a beep to a user when such a threshold has been reached.

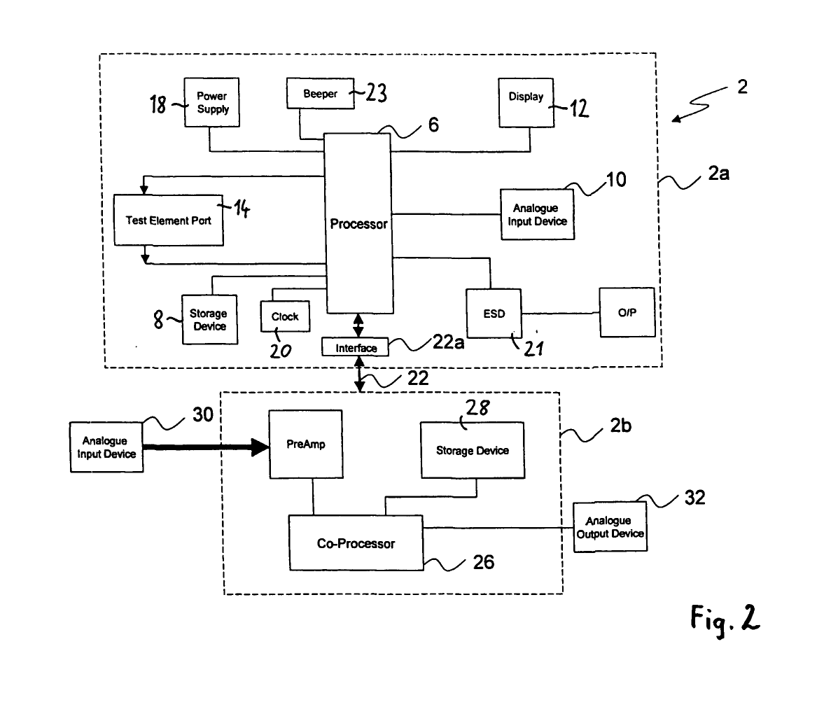

Further depicted in FIG. 1 is the second main element 2b. The second main element provides an alternative configuration for analogue input device, and is depicted in a simplified block diagram. Second main element is electrically connected by means of bus 22 to the first main element 2a. Alternative analogue input device may comprise a microphone circuit and/or a touch sensitive area(s) and/or additional switches. Whilst the first and second main elements 2a, 2b are described in the context of a shared integrated housing, it should be noted that the second main element 2b may be separate and external to the housing and connected to the first main element by means of the bus 22 cooperating with an interface 22a, as depicted in FIG. 2.

Data Reporting—Example 2

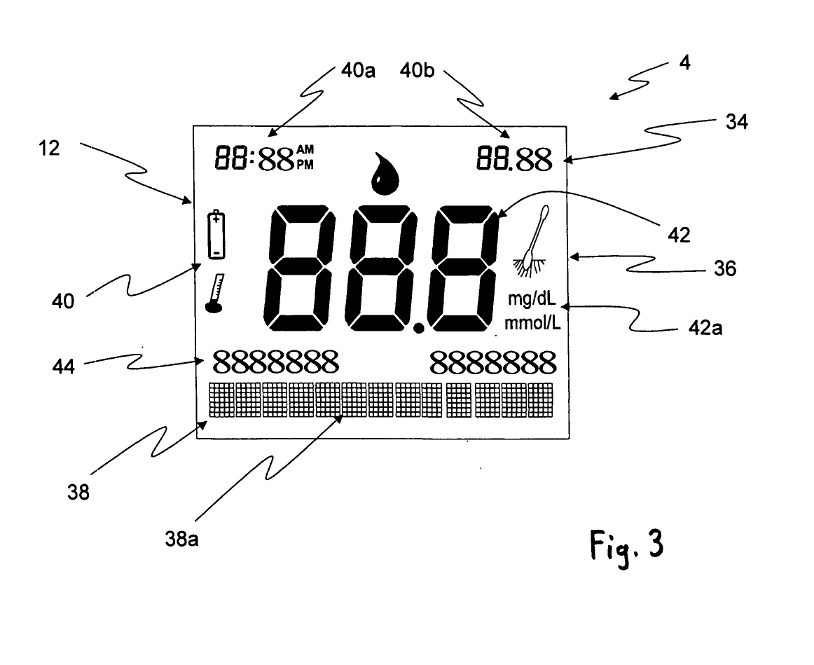

FIG. 3 shows, and admittedly in somewhat schematic fashion, an example layout of the display screen 12 forming part of the user interface 4 of the system. User operation of the device may be performed by navigating several different menu items as will be described in great detail later.

As shown in the current illustration, the display screen 12 forming part of the user interface 4 is divided into several sections (e.g. uppermost 34, middlemost 36, and lowermost 38) and arranged by way of example, for providing information to a user. Such divisions may include information icons 40, analyte measurement results area 42, and a lowermost information area 38a. The number of icons shown is purely by way of example and maybe varied to suit ones desires for performance of the device. As previously mentioned, the display screen 12 may be implemented by a commonly sourced colour or monochromatic dot matrix type, a segmented display and/or OLED display, and connected to an output port of the host microprocessor, as shown in FIG. 1. Preferably, the display screen 12 is provided as a hybrid segmented-dot matrix type.

The information displayed on display screen 12 is calculated based on data manipulation algorithms embedded within the host processor 6 for data analysis and statistical analysis. The algorithms may be pre-programmed on the host processor during device set-up and/or may be programmed on the host processor during device upgrade for instance. The parameters of the embedded algorithms can be updated or optimized by the user or by utilising the self-learning mode of the device. The utilisation of such algorithms is for allowing an easy, informative and convenient means of aiding the user in managing a condition for which the device of the present invention is being utilized e.g. for determining the concentration of an analyte (e.g. glucose) in a sample of physiological fluid.

Therefore, an uppermost section 34 of display screen 12 constitutes time 40a and date 40b indicators, utilising for instance, a segmented part of the hybrid display screen 12. The format of the displayed time 40a may be configurable by the user via navigation (as will be described in detail later) and selection of an appropriate menu item of the interactive navigational area of the interface program by means of the analogue input device 10, 30 such that a 12-hour or 24-hour format is assured. Similarly, indication of the date 40b may be in the form day/month/year or user configurable as year/month/day, again by selection of an appropriate menu item of the interactive navigational area of the interface program by means of the analogue input device 10, 30. General information icons 40 are further provided on the display screen 12 and as shown these may be provided as a ‘low battery’ icon 40c, a ‘temperature’ icon 40d, a ‘clean test element port’ icon 40e, and an ‘apply physiological fluid now’ icon 40f. Other icons may be provided as required or being specific to an analyte under investigation.

As previously mentioned, analysis of an analyte under investigation is a process undertaken by several components of the system e.g. test element, host processor, data analysis algorithm and so on, and provision is therefore made for displaying the results 42 of the analysis such that the user is informed instantaneously or near instantaneously of the resulting analysis. The result of the analysis may be reported to a user on a segmented area of the display screen. By way of example, the result 42 of the analysis is presented at the middlemost section 36 of the display screen 12, utilising 3×7 segments thus forming a numerical value. Generally, the segments utilised for displaying the analyte result 42 are of greater size than the segments utilised for displaying the time and date 40a, 40b information. The measurement results 42 may be stored in the form of analyte measurement result records on the host processor 6 and/or may be stored on a separate storage device 8 such as a ROM. Indeed it would be obvious to those skilled in the art that such resulting data may be transferred by wire or wirelessly to a personal computer for further analysis for instance.

Further displayed on the display screen 12 is the unit(s) of measure 42a for the analyte under investigation. As expected, the unit of measure 42a is allocated an area to the right of the numerical analyte result 42. Again, the unit of measure 42a may in part be user configurable such that accessing an appropriate menu item of the interface program would result in a preferred unit of measure 42a being displayed on display screen 12. In the case of glucose being the analyte, then the user may have the option of selecting between the units mg/dL or mmol/L. The unit of measure 42a may also be preset during manufacture depending on the geographical market destination of the device e.g. mg/dL being the preferred unit of measure in North America. Of course, different analytes require different units of measure and as such these are flavoured depending upon the utilised data analysis algorithm(s) hosted by the host processor 6. For instance, a device earmarked for evaluating coagulation properties of a physiological sample would require the unit of measure to be displayed as an International Normalised Ratio (INR).

Description of the Compliance Window and Additional Display Properties

As part of the compliance window a meal type identifier 44 is displayed on display screen. The meal type identifier 44 is a specific type of an event label of the compliance management profile, which indicates and links the management time frame for different meal events e.g. Pre-Prandial, Post-Prandial, occurring during the course of any 24 hour period of performed measurement values e.g. blood glucose values in case of a monitoring device for diabetes patients. The meal type identifier 44 may be displayed during the occurrence of an analyte measurement and/or may be displayed simultaneously when viewing a stored analyte result 42 record. As depicted in the current example, the meal type identifier 44 is provided as alphanumeric characters (i.e. text), that is by microprocessor 6 activation of appropriate segments and/or activation of appropriate pixels of the display screen 12. Similarly, non-alphanumeric characters may represent each meal type. In an alternative configuration (not shown), several meal type identifiers may be printed on a surface of a display viewing lens with an appropriate indicator activated on the display screen, juxtaposing the appropriate meal type identifier. Further, use of the system clock governs the displayed meal type, such that the indication of a meal type changes automatically during the course of any 24 hour period.

Further shown and by way of example only, on the lowermost section 38 of the hybrid display screen 12, is the lowermost information area 38a. The lowermost information area 38a is an area dedicated for delivering two forms of information. In a first form, provision is made for display information relating to analysis of data. In a second form, menu navigation information of the user interface of the device is provided. Navigation within the user interface of the device is realised by a multipurpose interactive navigational area 80 of the interface program which will be described in great detail later. Preferably, the lowermost information area 38a utilises a dot-matrix area under the control of the host processor to generate non-alphanumeric characters and alphanumerical characters.

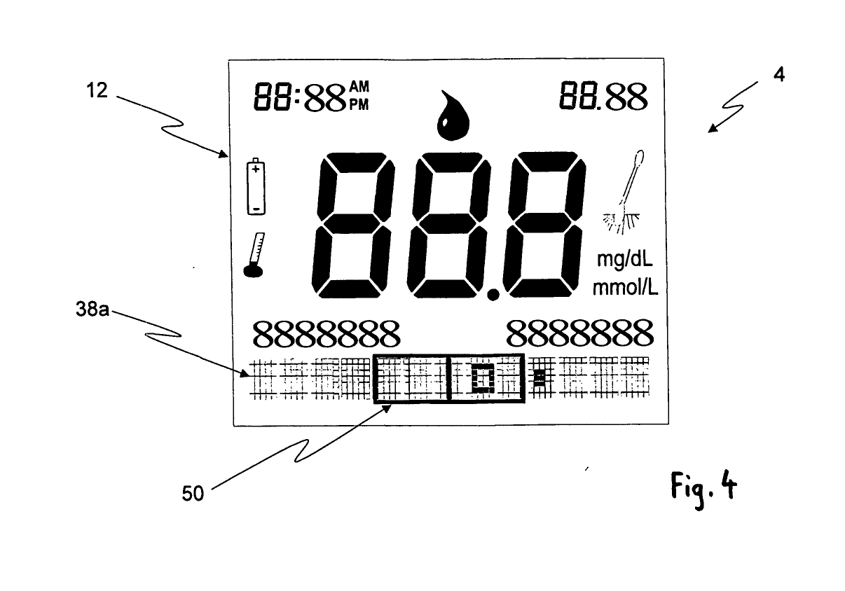

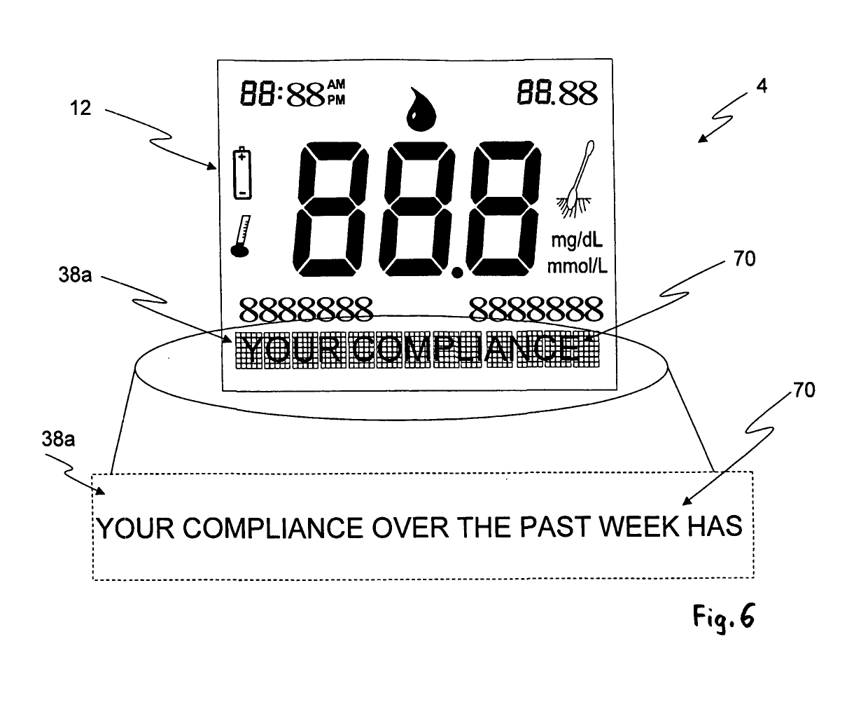

The data analysis information area 38a, in the first information form, is for providing an intuitive representation pertaining to user compliance to a treatment regimen. The representation may be alphanumeric and/or non-alphanumeric. In a non-alphanumeric form, the representation may be a bar graph and/or pie chart and/or any graphical or iconic way to display statistical summary of a disease state. Preferably, the representation is presented as a compliance window, as shown in FIG. 4. The representation, calculated using the aforementioned statistical and data analysis algorithms of the host processor 6, includes the provision of elements such that several graphical indicators are presented on the display screen 12 thus forming a disease management compliance window 50 for aiding a user in the management of a disease of interest. In an alphanumeric form, user compliance to a treatment regimen may be provided as text information and shown later in FIG. 6. Preferably, the text information scrolls from right to left across the width of the display screen.

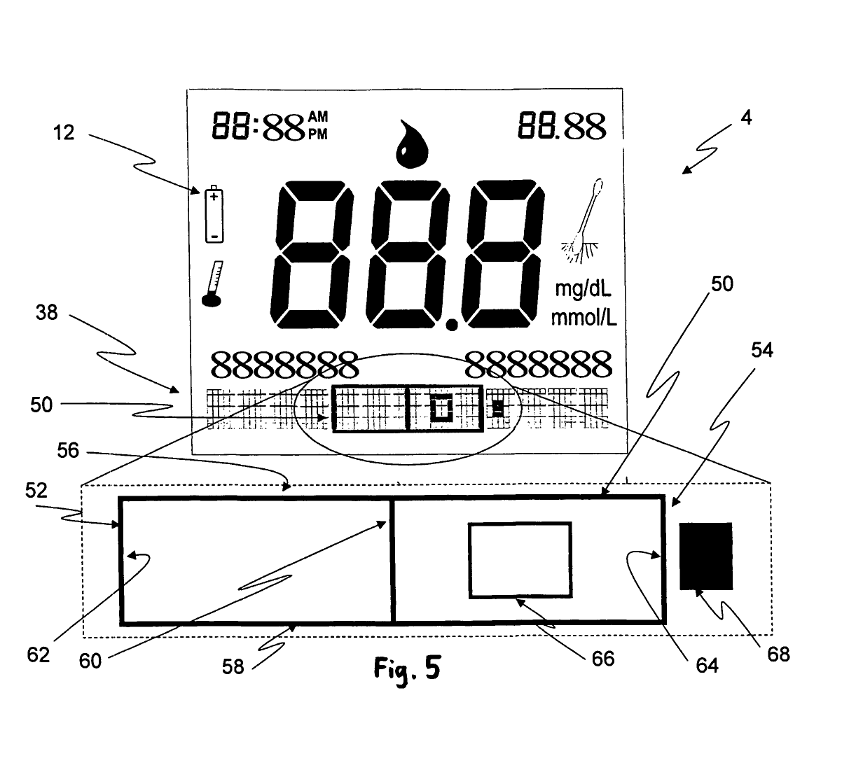

The disease management compliance window 50 may be defined as a geometric shape, by activated pixels and shown more clearly in FIG. 5. The geometric shape is provided by two pairs of opposing sides 52, 54, 56, 58, being of unequal length, defining a rectangular shape. In the current illustration, a horizontally aligned rectangle depicts the outer frame of the disease management compliance window 50 and as previously discussed arranged on the lower most section 38 of the display screen 12. It would be obvious to those skilled in the art however that the window 50 may be in the form of a circle, a triangle, a square and so on, and disposed on any area of the display screen 12.

The disease management compliance window 50 further includes several activated pixels forming several category indicators. The category indicators represent several different information categories. Such information categories may represent a ‘target indicator’ 60, and/or a ‘low and high threshold indicator’ 62, 64 and/or a ‘mean indicator’ 66 and/or a ‘current measurement indicator’ 68, and/or any other indicator deemed relevant to aid a user in the management of his disease. The indicators may be arranged vertically, and/or horizontally, and/or diagonally, although one of skill in the art, given the teaching of this invention, will recognize that deviations from this may be accomplished without varying from the scope of the present embodiment. In the case of a horizontally aligned rectangle being utilised as the disease management compliance window then it is preferable that vertically arranged indicators are utilised. It is anticipated that the disease management compliance window is not limited to a glucose analyte, and many several other analytes e.g. cholesterol, tri-glycerides, lactate, or drugs with a narrow therapeutic range i.e. Coumarin as well known as Coumadine® or Warfarin® and so on could validly use such a window utilising appropriate and/or specific compliance information and feedback to the user. The aforementioned can be displayed either as a graphical compliance window or with aid of category indicator(s).

The disease management compliance window 50 may allow a user to visually compare an indicator (or icon) representing a current measurement 68 with indicators (e.g. icons) representing threshold 62, 64, target 60 or mean measurements 66, based on their appearances and locations about the compliance window. According to the comparison the user may elect to modify his lifestyle (e.g. increase exercise, reduce certain food types and so on), to make it more likely that a current measurement indicator 68 will closer correlate with one or more of the other indicators. The compliance window 50 may display information that changes in substantially real time as the current measurement indicator 68 moves within or outside of the compliance window 50 to provide a substantially accurate display of the values associated with the indicators at all times, for example in the case of a continuous analyte (e.g. glucose) sensor.

Generally however, the upper and lower compliance threshold indicators 62, 64 are represented by one pair of opposing sides 52, 54 of the disease management compliance window 50 i.e. the vertically arranged sides. As will be described in great detail later, the location of the indicators (e.g. thresholds, targets) relative to the disease management compliance window 50 may be predetermined by the user or HCP during device set up, utilising the analogue input device in form of buttons and/or switches 10 and/or touch sensitive area(s) 30 which does not require a mechanical activation for selecting an appropriate menu item of the interactive user navigational area. Further, a numerical value 42 (e.g. indicative of the concentration of an analyte for each indicator) may be presented to the user during access and operation of a compliance window 50 set-up mode. The numerical value 42 may be presented on the middlemost section 36 of the display screen 12 utilising 3×7 segments or such values may be presented utilising the segments partly reserved for displaying the current date and time information.

Further shown, and forming part of the disease management compliance window 50, is the analyte measurement target indicator 60. The position of the target indicator 60 is again predetermined by the user and/or HCP and it is generally considered that such an indicator 60 shall be positioned within the disease management compliance window 50, that is between the lower and upper threshold indicators 62, 64. In the current illustration, the pixels activated to display the target indicator 60 are depicted and arranged as a vertical line, being aligned parallel to the lower and upper threshold indicators, although it would obvious to those skilled in the art that deviations from this may be accomplished without varying from the scope of the present embodiment. As will be described later, the user may alter the position of the target indicator 60 depending on his health status and/or may be prompted, by means of the piezo transducer (beeper) 23, to alter the target indicator at a predetermined frequency i.e. once a month.

The provision of a mean indicator 66 allows for representative indication of all analyte measurements in relation to a specific meal type 44 e.g. pre-prandial, post-prandial occurring during the course of any 24 hour period. Similarly, other category indicators (e.g. current measurement) are activated in dependence of the embedded data and statistical analysis algorithms such that the x axis (and/or y axis in the case of vertically arranged compliance window) of the activated pixels (i.e. indicators) are correctly positioned about the compliance window 50. That said, the aforementioned category indicators are thus arranged in the context of one another and ‘move’ depending on a result of several factors such as the calculated analyte result for instance.

In the case of the mean category indicator 66, the x (and/or y) co-ordinate(s) thereof in relation to the target 60 and the threshold category indicators 62, 64 are determined by a data analysis algorithm embedded on host processor, in this case as Equation 1.

$${Analyte}_{mean} ( \overline{x} ) = \frac{1}{N} {\sum }_{i = 1}^{N} {x}_{i} \qquad Equation 1$$

Where Analyteₘₑₐₙ is the mean analyte value of all x measurements over a time frame of interest, and N is the number of data points (or measurement).

According to a preferred embodiment, the mean analyte value is determined for one specific meal time category, like e.g. “pre lunch” or “after dinner”. For determining a mean value for the meal time category “pre lunch”, only former measurement values of this specific meal time category are taken into account. Thus, specific mean values corresponding to each of the meal time categories may be determined.

Preferably, the respective mean values are determined as mean values of the measurements of the last n days, and further preferably, n is set to 14, which means that an average value of the measurements of the last 14 days is determined for a respective meal time category. In a further preferred embodiment, each time a test result is calculated, a new average for that mealtime is also calculated, with the new average being stored together with that result.

In the current illustration, the location of the mean category 66 indicator is at a position between the target and upper threshold indicators 60, 64. Indeed it is generally considered that effective disease management ensures that a mean category indicator 66 is as close as possible to a target category indicator 60. The mean value for each specific meal type frames may be stored in the storage device of the first main element, such that the value(s) may be utilized for subsequent statistical calculations and/or recalled for presentation to the user in the future such as for comparison with other date specific time events.

Depicted next to the upper threshold category indicator 64 is the current measurement indicator 68 i.e. the indication of an analyte measurement relative to a target 60 and/or mean indicator 66. Indeed, in the current illustration, the current measurement indicator 68 may be provided by several activated pixels forming a geometric shape (e.g. square, circle, triangle, and so on) or an icon, although one of skill in the art, given the teaching of this invention, will again recognize that deviations from this may be accomplished without varying from the scope of the invention. The position of the current measurement indicator 68 relative to the target measurement indicator 60 is, of course a direct consequence of the concentration of an analyte under investigation and therefore determined by an analysis algorithm hosted on the host processor. An alarm may additionally be triggered to alert the user that a current measurement (or a series of measurements) is/are outside of the upper and/or lower thresholds. Such an alarm may be audible 23 (by means of the beeper), iconic 40 (by means of the display), or vibratory (by means of a vibrator) (not shown).

According to a preferred embodiment, each time a test result is calculated, a new average for that mealtime category is also calculated, and the new average is stored with that result. The monitoring device displays the last record made for that mealtime category, like e.g. “before breakfast”, “after breakfast”, “before lunch”, “after lunch”, etc. The user may scroll back through the records for that mealtime category, one result at a time. When the user scrolls through the records for that mealtime, he or she will see the mean indicator 66 move within the compliance window 50. The user sees that the mean indicator 66 moves towards the target 60 or away from the target 60. The monitoring device displays the records in the selected mealtime category. The mean indicator 66 indicates the 14-day average at the time when the record was made. The user can see the 14-day mean indicator 66 move within the compliance window as he or she scrolls backwards and forwards through the records a in the selected mealtime category. The movement of the mean indicator 66 indicates to the user whether his or her lifestyle is correct or incorrect.

Such a trending function is particularly useful to be displayed in combination with the averages of the different categories to indicate to the user his on-going compliance state and/or states.

For evaluating a measurement result, the monitoring device will use simple routines to make comparisons between elements of a record, using the compliance window settings as benchmarks. In particular, the following three comparisons may be performed:

First Test: Comparing the Target Value with the Updated 14-Day Average

The target value is set as one of the compliance window parameters. This value will not vary from record to record unless the user adjusts his or her personal parameters. This is also true for the low limit and high limit parameters. Every time the user makes a new blood glucose measurement for a particular meal memo category, the monitoring device will use the new result to calculate a revised 14-day average. The average value is stored as part of the glucose result record which means that each record contains a 14-day average value that was true at the time the measurement was made. This provides a simple trending tool that allows the user to see through a graphical display how his or her average is changing over time. This feature is further enhanced by making a basic numerical comparison between the 14-day average value and the target. If the net difference between the two values is greater than a pre-set value, say ±30 mg/dL for example, the monitoring device will display an advice screen like e.g. “YOUR AVERAGE IS TOO LOW COMPARED TO YOUR TARGET” or “YOUR AVERAGE IS TOO HIGH COMPARED TO YOUR TARGET” or “CONSULT YOUR DIABETES ADVISOR”.

Second Test: Comparing the Target Value to the Current Result

A simple numerical comparison between the result just calculated and the target value can be made. If the net difference between the two values is greater than a pre-set value, say ±50 mg/dL for example, the monitoring device will display an advice screen like for example “YOUR RESULT IS LOW COMPARED TO YOUR TARGET” or “YOUR RESULT IS HIGH COMPARED TO YOUR TARGET”:

Third Test: Comparing the Result to the New 14-Day Average Value

A simple numerical comparison between the result just calculated and the new 14-day average can be made. If the net difference between the two values is greater than a pre-set value, say ±50 mg/dL for example, the monitoring device will display an advice screen such as for example “YOUR RESULT IS LOW COMPARED TO YOUR AVERAGE” or “YOUR RESULT IS HIGH COMPARED TO YOUR AVERAGE”.

Optionally, an indication of the variance (not shown) of analyte measurement over a selected time frame may additionally be displayed about the compliance window.

Data Reporting—Example 3

The data analysis information area has, so far been described in relation to non-alphanumeric representation of user compliance of a treatment regimen, and as such to further aid the user to understand his compliance to a disease, the provision of alphanumerical information, in the form of scrolling text 70 or messages, may be provided. As illustrated therefore in FIG. 6, the lower most information area 38a is utilised to provide the scrolling text information 70, by rapid host processor 6 activation and deactivation of appropriate pixels. The scrolling text 70 may further be provided elsewhere about the display if a full dot matrix display screen is utilised. The messages 70 exhibited on the display screen 12 may be pre-programmed and stored on the host processor 6 or on the storage device external 28 to the host processor 6, providing useful information to a user. Accordingly, the exhibited message may relate to compliance of a treatment regimen, and/or advertisements from sponsors, and/or company logos, and/or motivational messages, and/or personal information (e.g. Name, allergies, contact person and so on) and/or general information relating to a disease for which the device is implemented. Desirably, the provision of the messages 70 relating to disease management compliance information is automatically prompted in relation to measurement values and their comparison to predetermined thresholds, as will be described below. Further, a provision is made for user control of the rate of text scrolling such as by selection and manipulation 10, 30 of function menu items of the interface program.

An example pre-programmed message which may be conveyed to a user in a scrolling fashion may be ‘YOUR MEAN GLUCOSE VALUE DURING BREAKFAST IS ON TARGET’. Similarly, a message indicating an elevated glucose levels between different meal types could be displayed such as ‘YOUR MEAN GLUCOSE VALUE DURING BREAKFAST IS HIGHER THAN DINNER’. Indeed, such a message may additionally be conveyed to the user by means of the analogue output such as a speech digitizer and a speaker.

Whilst an indication of mean analyte measurements is an important parameter in aiding a user during management of a disease, it does have limitations in the information that is presented to the user. Potentially, there is a possibility that the user may experience dangerous high or low glucose concentrations, so a measure of how far the measurement values are spread apart might be considered useful. For instance, a diabetic patient testing his glucose concentration during a breakfast time frame and over the course of five days might have a mean value of 8.6 mmol/L. However, such a value does not truly reflect the full range of results over such a time period. Consider a diabetic patient with a range of results of 4, 3, 13, 15 and 8 mmol/L, and contrast with a range of results of 8, 9, 10, 7, and 9 mmol/L for the same time frame. Clearly the former set of results has a large spread compared against the latter, although the mean in both cases is 8.6 mmol/L.

Therefore, further statistical analysis of stored data may provide the user with much more meaningful information with regard to disease management compliance. In one aspect of the present embodiment, standard deviation analysis may be provided by utilization of Equation 2. Briefly, the equation describes the standard deviation of measurements over a predetermined time frame. Using Equation 2, the standard deviation of measurement values can be calculated for indicating the spread of data about the mean value. For example using the above examples would return a standard deviation value of 9.8 for the former set of results and in turn, a standard deviation of 2.3 for the latter set of results. The result of using Equation 2 therefore indicates the spread of the measured values for each meal type suggesting that the former set of results are variable.

The standard deviation equation forming part of the statistical analysis is given by:—

$$stv = \sqrt{\frac{\sum {( x - \overline{x} )}^{2}}{N - 1}} \qquad Equation 2$$

Where x̅ is the mean (from Equation 1), x is the glucose value for each data point and N is the number of samples.

Even more powerful is the possibility of a comparison between the spread of data of at least two separate data sets (e.g. meal types, i.e. breakfast, lunch, and pre and post prandial) that have approximately the same mean. For example, the mean value during breakfast measurements might be the same (or near) as the mean value for the dinner measurements, potentially mis-leading the user that preceding actions (e.g. exercise, diet) has been conducive in providing disease management compliance information, or that both data sets are the same. However, by calculating the standard deviation and presenting a message to the user, driven by such results, it would become apparent to the user that the breakfast measurements may have high variability of measurements as compared to the dinner measurements. For example, a relatively small standard deviation, in this case 2.3 is indicative of a narrow spread of measurements around the mean and therefore has comparatively fewer high or low value measurements. Conversely, a relatively large standard deviation, in this case 9.8 is indicative of a wide spread of measurement around the mean. Generally, the more widely spread the values are, the larger the standard deviation. Further, the high spread of data during breakfast time frames might indicate that his previous insulin dosage may be too high or low. The resulting analysis may be provided to the user by means of scrolling text with such a message being ‘YOUR BREAKFAST READINGS ARE VARIABLE AND SOMETIMES OUT OF THE NORMAL RANGE. PLEASE CONSULT YOUR PHYSICIAN’.

In practice, the standard deviation for each meal type is stored in the storage device 8 as a running value, after the user has performed an analyte measurement. Similarly, a comparison is performed by operational software of the host processor 6 of outlying returned results, that is, results of Equation 2 which provide the largest range. For instance, if four standard deviation results (i.e. corresponding to four different meal events) are computed using Equation 2, then the results of the comparison may be utilised to return a message to a user (e.g. as pixelated scrolling text). Messages returned to the user may include ‘GLYCEMIC CONTROL DURING BREAKFAST HAS A LOT OF HIGH AND LOW VALUES COMPARED TO OTHER MEAL TIMES. IS YOUR DOSED BED TIME INSULIN LEVEL APPROPRIATE?’

Further, a comparison could be made between same time frames to allow the user to judge if his analyte measurement values are within predetermined thresholds, or that the results over the same times frame are variable or not. In this case, the user may be presented with a statement such as ‘YOUR GLYCEMIC CONTROL AT BREAKFAST LAST WEEK WAS MUCH BETTER THAN NOW. HAS ANYTHING CHANGED?’

Further, trends of a patient compliance to a treatment regimen may additionally be provided such that a determination is made whether an overall increase (or decrease) in the control and variability of the measurement values over a predetermined time frame. Messages may be provided to the user and displayed in text box such as ‘OVER THE PAST MONTH YOUR DINNER GLUCOSE VALUES ARE MUCH BE TIER CONTROLLED’, thus providing a motivation message to the user.

Data Reporting—Example 3

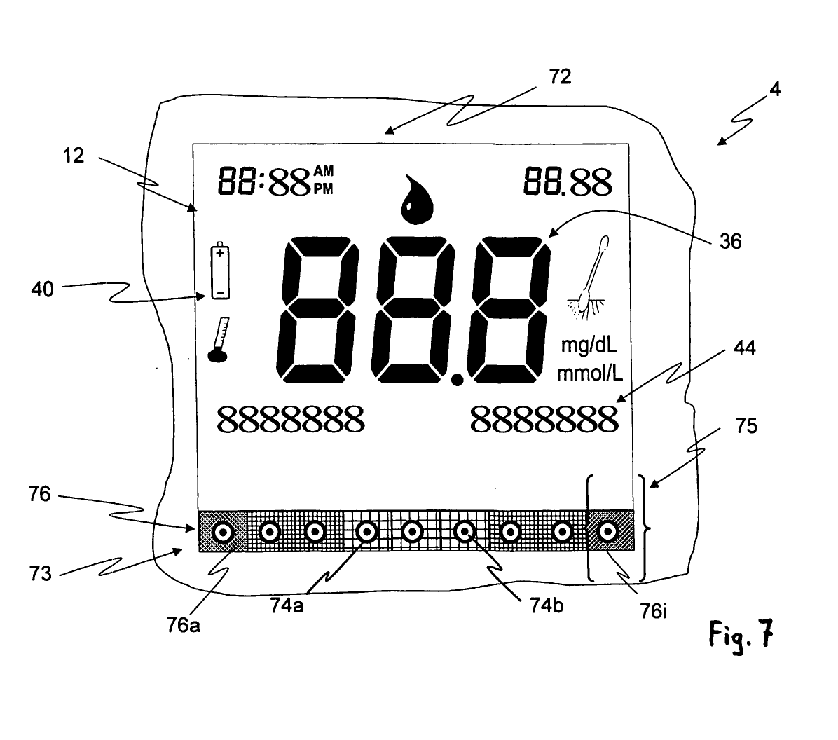

In another embodiment of the present invention, disease compliance information is presented to a user in a somewhat simplified manner. FIG. 7 therefore shows and again in a somewhat schematic fashion, an alternative configuration of the disease compliance reporting aspect of the user interface. The figure shows a display lens 72 positioned above a display screen 12 and within an aperture of an upper housing of the device.

Similar to the illustration in FIG. 3, the display screen 12 is divided into several sections or areas and arranged by way of example, for providing information to a user. Again, such divisions may include information icons 40, a results data area 36, and meal event indicators 44. Further provided, preferably on the lowermost part of the display screen 12 are icons 74, earmarked for disease compliance notification. Again, the display screen 12 may be implemented by a commonly sourced colour or monochromatic dot matrix type, a segmented display and/or OLED display, and connected to an output port of the host processor 6, as shown in FIG. 1. In the present embodiment, the display screen 12 is provided as a segmented type.

The display lens 72, being transparent in nature, may be of various sizes and shapes and includes an inner surface and an outer surface. The display lens 72 may form part of the outer casing (not shown) of the device or may be connected, by means of snap fitments for example, to the outer casing of the device. Such connection generally is for providing a contiguous fitment between an outer surface of the display lens 72 and an outer casing of the device.

Either the inner and/or outer surfaces of the display lens 72 may be coated with a coating having antireflective properties, such as that undertaken by a chemical and/or physical process as would be known to persons skilled in the art. Such properties aid a user when viewing the contents of the display screen 12 under bright light conditions e.g. sun, and/or lamps. Further, a non transparent circumferential border 73 may be provided by printing or coating of the inner surface display lens 72 with a non-transparent material.

On a lowermost aspect of the border 73, a cell matrix 76 is provided comprising of individually separated geometric shapes or cells 76a-i and preferably defining a graduated chromatic scale (e.g. colour or mono), by further printing or coating techniques. In a preferred embodiment the chromatic scale is provided behind the glass of the liquid crystal display (LCD) with individual activated segments of the display appearing in front of the chromatic scale. Each individual geometrically shaped cell 76a-i may however comprise an individual patterned marking. The scaling may vary in a linear or non-linear manner in order to cause the graded effect to be accentuated or attenuated. Preferably, a 9×1 cell matrix 76 is utilized, with the cells 76a-ic being rectangular in shape. However, cells 76a-i may have any suitable size and shape according to particular needs. The cells 76a-i may be disposed elsewhere about the border 73 such as the uppermost and/or on the sides and arranged either horizontally or vertically. Preferably, the cells 76a-i are clustered around the lowermost peripheral border 73 in a horizontal arrangement that together with suitably activated symbols 74a, 74b provide a compliance indicator, as will be described below. Cells within the matrix are scaled such that in combination with the activated symbols represent ‘ELEVATED’, and/or ‘SLIGHTY ELEVATED’ and/or ‘NORMAL’ current measurement and/or statistically enhanced measurements (e.g. mean).

The number and form of the symbols 74a, 74b depicted in the current illustration are purely provided by way of example and varied to suit ones desires, however the number of symbols 74a, 74b available for display on the display screen 12 is equal to twice the number of cells 76a-i the matrix 76. The symbols 74a, 74b may be provided by way of segments (e.g. in a segmented type display, and pixels in a hybrid and/or dot-matrix display). Alternatively, pixels that define alphanumerical and/or non-alphanumerical characters may be utilised if a pixelated type display is utilised. As mentioned it is preferable that matrix contains nine cells, with nine pairs of concentric circles provided. Each circle within a pair are distinguished by having an inner circle and outer circle of different diameter and separated by a gap. Each circle may be independently activated.

Each activated symbol 74a, 74b (i.e. circle) may be representative of statistical evaluations performed by the host processor 6. For example, an inner activated circle may represent a ‘current’ measurement, whereas an outer activated circle may represent a ‘mean calculated value. Such an outer circle may be activated and controlled by Equation 1. Furthermore, the position of each circle in relation to the cell matrix is dependent and/or activated depending on predetermined analyte measurement targets. Such targets may be user defined and/or HCP defined, each user accessing a set-up mode of the interface program by manipulation of the analogue input device 10, 30.

The activated symbols 74a, 74b on the display screen appear in front, in a preferred configuration, of the display lens cell matrix 76. Such a relationship defines compliance indicator 75, enabling a user to readily visualize and compare among the cell markings 76a-i the different status of his compliance to a disease regimen, without needing to conduct a detailed evaluation of the actual values associated with the analyte measurement as in known techniques.

For instance, an activated inner circle 74a on the display screen 12 appearing in front of a coloured cell 76a or 76i (e.g. red) may indicate to a user that his current analyte measurement is ‘ELEVATED’. Further, an activated outer circle 74b appearing in front of a coloured cell 76d-f (e.g. green) may indicate that his mean analyte measurement for a particular meal type ‘NORMAL’. Indeed, indication of a specific time frame is also provided on the display screen 12, further enhancing the information provided to the user.

Users who improve their compliance to a treatment regimen may suitably alter their targets, again by accessing an appropriate menu item of the interface program and manipulation of the analogue input device 10, 30.

Whilst the present embodiment has been described in relation to geometrically shaped cells 76a-i provided on the display screen, it would be obvious to those skilled in the art that such geometrically shaped cells 76a-i may easily provided on a surface of the display lens and/or casing of the device. In the latter, such activated symbols are envisaged to be in a juxtaposing relationship to the display screen 12. Further obvious to those skilled in the art would be provision of disease compliance indicators 75 formed by utilisation of a colour display screen e.g. colour pixel display screen, such that a cell matrix is formed by appropriate pixels. Indeed further obvious to those skilled in the art, would be provision of at least one light emitting diode (LED) on the device, again activated by the host processor to represent the effect of the compliance indicators. The LED may be a tri-colour type, a flashing type, an array, or a combination thereof.

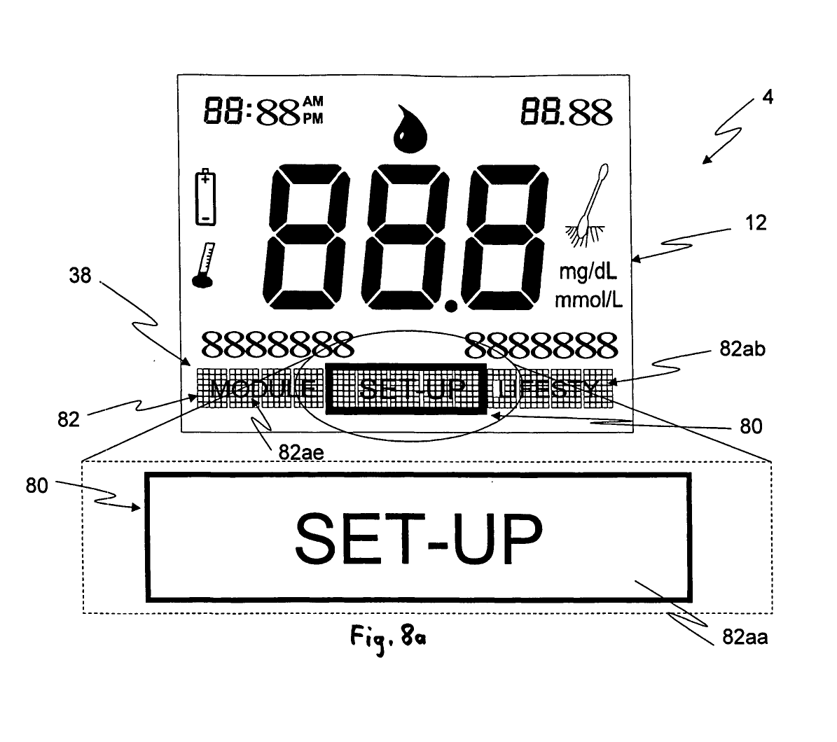

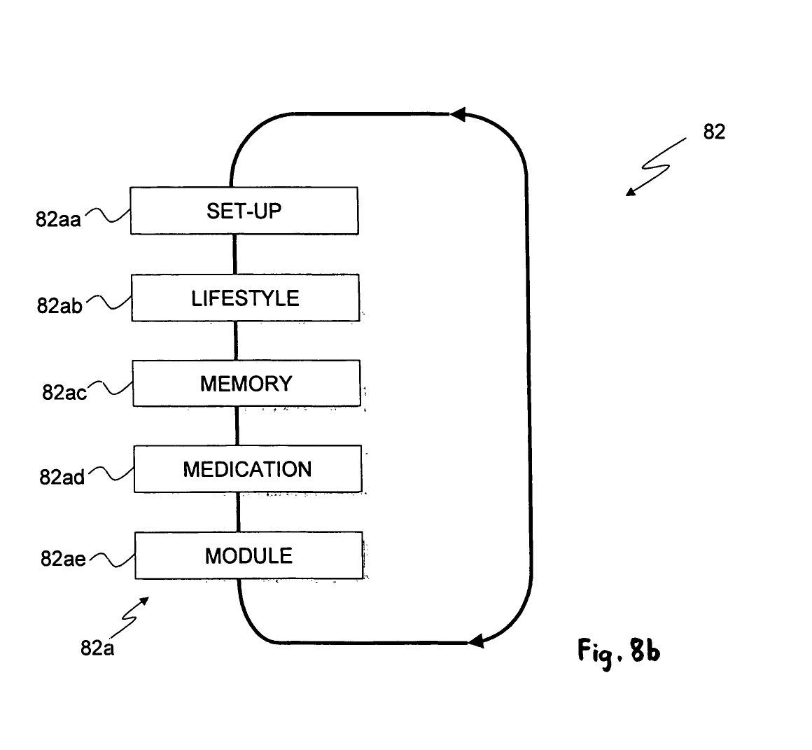

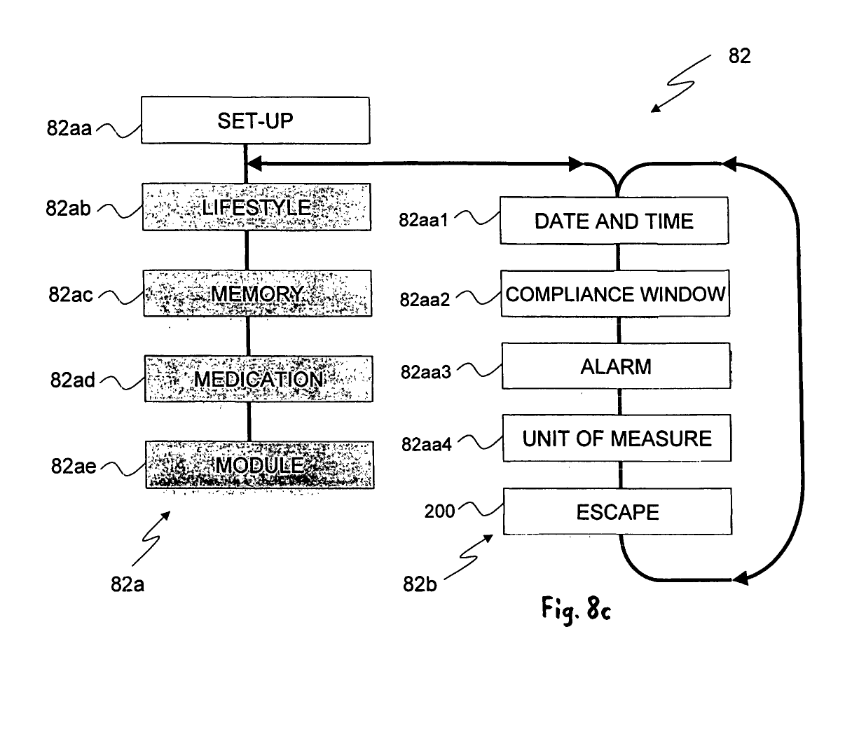

Whilst the aforementioned embodiments are particularly directed to the manner in which measurement data are presented to the user and providing the user with a synopsis of his compliance to a treatment regimen, the manner in which the interface program operates in particular reference to FIGS. 8a-8c will be described in detail below.

Example for the Interactive Navigation Area

In FIG. 8a, the host processor by means of the interface program, is configured to present a navigational interface on the display screen 12 to a user. The navigational interface comprises a menu interface 82 such as a list menu and a navigation decision area.

In an exemplary implementation, the navigation decision area is provided by a ‘selection window’ 80, and provided by the host processor 6 on the display screen 12 by several activated pixels. Preferably, the selection window 80 is displayed at the lowermost section 38 of the display screen 12, that is the lowermost information area. The selection window 80 may be provided as a geometric shape having an outer frame provided by the activated pixels. The geometric shape may be in the form of a circle, a triangle, a square, a rectangle. Preferably, a horizontally aligned rectangle is provided, although it will be apparent that the selection window 80 can be readily arranged in any other orientation on the display screen 12. Of course, apparent to those skilled in the art would be the provision of a selection window 80 on a surface of a display lens 72, provided by means of a coating for example.

Although not shown in the current figure, provision is made for the analogue input device 10, 30. As mentioned previously, the analogue input device 10, 30 can take the form of control switches 10, touch sensitive areas 30, jog wheels.

User input to the host processor 6 may be effected by manipulation (or instructing) of the analogue input device 10 (e.g. manual buttons, confirmation button) which in turn signals the host processor 6.

Generally, the list formation of the menu consists of a set of individual menus logically organized in a hierarchical fashion, such that the selection of a first level menu item from a first level menu causes a second level menu to be displayed. The second level menu, in turn, includes several second level menu items associated with the selected first level menu item. Although two menu levels are described in the exemplary implementation, it will be apparent to those skilled in the art that further menu levels can be easily added. Menu items at any level in the hierarchy can be provided to cause another menu level to be displayed, to set targets or thresholds, to view stored analyte measurement result records, or to cause a particular function to execute. The menu 82 of the present invention is described herein with respect to an exemplary implementation that allows a user to select a function from several available functions.

FIG. 8b shows a top level menu 82a which may be presented on the lowermost information section 38 of the display screen 12. The top level menu 82a includes a set of top level menu items 82aa-82ae in a sequential arrangement. While five menu items are depicted, it should be recognised that a greater or lesser number of menu items can be used without departing from the scope of the present embodiment. As will be described herein, a user selected menu item (82aa of FIG. 8a) is set within the selection window 80. Further, and at any time at least three menu items are displayed on the display screen 12 arranged in a sequential arrangement (e.g. row or column). The menu items are provided in the context of the selection window 80, such that manipulation of the analogue input device 10 scrolls the menu items 82aa-82ae (of FIG. 8b) in a left or right direction (or up and down), depending on the direction in which the user urges the analogue input device 10 such that a required menu item 82aa is positioned within the selection window 80 as shown in FIG. 8a. Further, either side of the selection window 80 is a pre and post menu item 82ab, 82ae, for providing a preview (or post view) of the next menu item a user may be able to select.

Each menu item represents a link to another menu level, a feature, an application, and so on. Further, each menu item includes text and/or a graphical icon to present the function of the link. The text and/or graphical icon may be represented by pixels under the control of the host processor 6. In the present example, the links are labelled in the following categories: ‘set-up’ 82aa, ‘lifestyle’ 82ab, ‘memory’ 82ac, ‘medication’ 82ad, ‘module’ 82ae. The menu items maybe arranged according to a default configuration as may be specified by a manufacturer. Indeed, further first level menu items may be added to the first level menu 82a on connection to the device of appropriate functional hardware modules. Similarly, first level menu items seldom used by user may be ‘removed’ therefrom. Such operations are performed by accessing first level items such as the ‘set-up’ item.

Upon selection of a first level menu item 80aa from a first level menu 82a, a second level menu 82b is generated and displayed on the lowermost section 38 of the display screen 12, shown in FIG. 8c. The second level menu 82b comprises several second level menu items, and the second level menu items replace the first level menu items in the row (or column) arrangement. Again, at least three second level menu items may be displayed on the display screen 12, one of which being inside the selection window 80. The remaining second level menu items appear either side of the selection window 80 allowing a user a pre or post view of such menu items. FIG. 8c depicts an example of a second level menu displayed in response to a selection of a ‘set-up’ menu item 82aa (of the first level menu 82a). Accordingly, the second level menu 82b includes several second level menu items 82b, arranged in a row (or column) arrangement. Again, all of the second level menu items 82aa1-82aa4 include text and/or a graphical icon to represent the function of the item. Further, selection of any second level menu items 82aa1-82aa4, as in the selection of items from the first level menu, utilises other parts of the display screen 12 such that stored records, set-up information (e.g. time, date, targets), optional module information and so on are displayed on the uppermost or middlemost area of the display screen. Further provided is a means 200 of navigating back from the second level menu to the first level menu or from the first level menu to the main screen. Such means 200 is provided by an “Escape” or a “Go-Back” menu item.

Selecting a menu item can be accomplished in a number of different ways. Referring to FIG. 1, in one example, the menu item can be selected by navigating through the menu items of each menu by the analogue input device 10 until the desired menu item appears in the selection window. In practice, assume that the ‘memory’ menu item 82ac is within the selection window 80 and the user desires to navigate into a second menu level of the ‘set-up’ menu item 82aa. To do so, the analogue input device 10 is urged by the user in a general left or right direction, causing the menu items to move in a corresponding fashion through the selection window 80, until the required item (i.e. set-up) is within the window 80. The desired menu item is then selected by the user by performing an action e.g. depression of the confirmation switch. As previously mentioned, the analogue input devices i.e. control switches, includes text and/or graphical icons which may be backlit by a light source (e.g. LEDs) for aiding a user during low light conditions. The second menu level 82b is then entered and associated further menu items are presented on the display screen. Further urging and depression of the input device 10 allows the user to select the desired menu item. It should be appreciated that as the menu items are selected and different items or functions are entered, the information provided on other parts of the display screen are changed as appropriate. For example, in the memory menu 82ac, previous generated analyte results 42 can be viewed on the display screen 12 (e.g. upper and middlemost area) by control of the analogue input device 10. The previously generated analyte result records (e.g. for each meal event, or particular date) can be viewed in a sequential fashion, the desired record being viewed by appropriate urging of the analogue input device 10.

Description of meal marking in relation to a Measurement Sequence

As previously mentioned, the disease management compliance window is a graphical tool aiding a person to visually compare an indicator such as for a current measurement value with other indicators deemed important for the management of a disease. These other indicators may include high and low markers and/or average markers and/or other statistical relevant markers. With this in mind, it is important to note that in the context of diabetes management, diabetic patients attempting to regulate their glucose values with the aid of self monitoring glucose meters must be provided with analytical tools to achieve such control. Such tools are particularly useful for newly diagnosed patient who may be inexperienced with disease management. Subsequently, for the user to glean useful information from the disease management compliance window, it is imperative that glucose measurements are classified or categorised into different meal events e.g. pre-prandial and post-prandial so that any supporting tools (be it graphical based or not) work in partnership with such information.

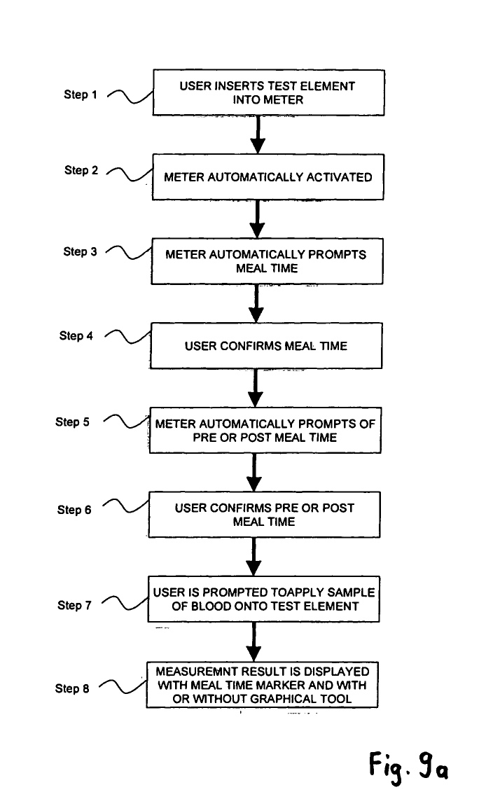

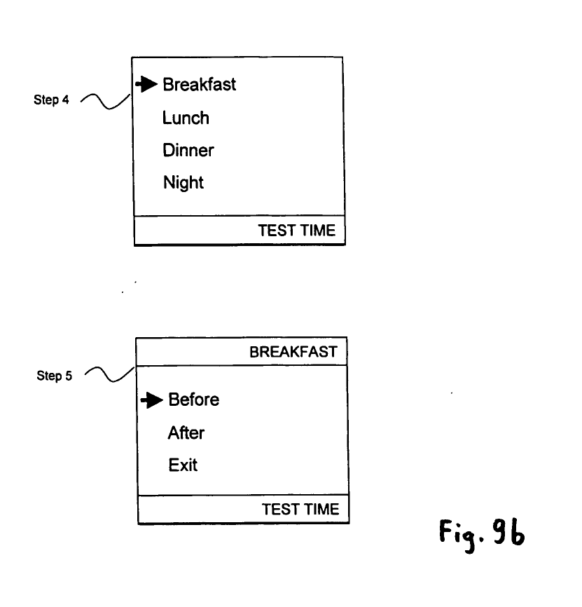

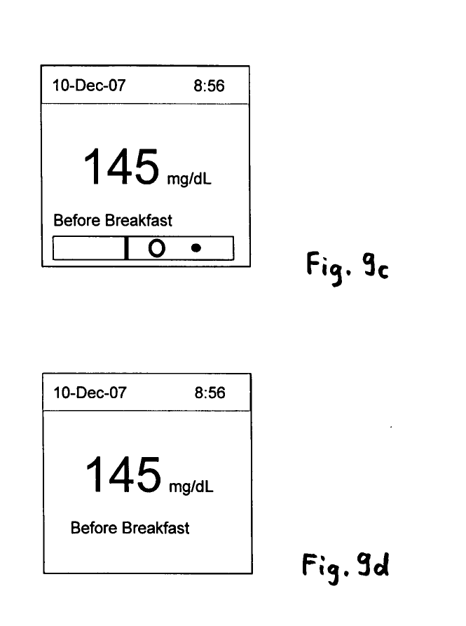

Turning now to FIG. 9a, the figure shows a flow chart showing the sequence of performing a blood glucose measurement with the disease management compliance window optionally activated. In a first step (Step 1), the user inserts an unused test element into an appropriately designed receptacle. Such a receptacle may be configured to receive a photometric based test element such as the analyte test element of European Patent Specification 1574858, or the receptacle may be configured to receive an analyte test element of Co-Pending Patent Application PCT/EP2004/009113. After insertion of the test element by the user into the receptacle, the meter is automatically activated (Step 2), that is to say the previously mentioned electronic circuits are switched from a stand-by state to an on-state. In a next step (Step 3), and after the meter is activated, the display screen of the meter prompts the user to select a meal time from a pre-programmed list of possible meal time categories (e.g. Breakfast, Lunch, Dinner, Night, Other). The list may be in the form of text based messages and/or iconic based messages and/or a combination of text and iconic based messages. By actuating a switch (e.g. mechanical and/or touch sensitive and/or voice control), the user selects and confirms an appropriate meal time category to conclude the next step (Step 4). To reduce the number of steps the user needs to interact with the meter, the automatic prompting of the meal time is based on operating parameters such as e.g. 6 am to 11 am equating to ‘Breakfast’, 11 am to 3 pm equating to ‘Lunch’, 3 pm to 8 pm equating to ‘Dinner’, and any other time equating to ‘Night’ or ‘Other’. Such operating parameters may be stored on a storage device of the meter for example. It is important to stress, that these operating parameters are not fixed and that the user can select which ever meal event which is most apt for a particular time of day.

Next, (Step 5) the content of the display screen is automatically changed to display a further user selectable list. The changes to the display screen is to allow the user to select whether the previously selected meal category should be marked as pre or post prandial type. The list may be in the form of text based messages and/or iconic based messages and/or a combination of text and iconic based messages. For example, the display screen may indicate the previously selected time, in this case, ‘Breakfast’ in a header part of the display and in the centre thereof the user selectable pre and post list (‘Before’ or ‘After’), as is shown in the schematic diagram of FIG. 9b. In a footer part of the display screen, is an information area to indicate which menu level and/or action the user must perform.

In a next step (Step 6), the user categorises the previously selected meal category into a pre or post prandial type by navigating to the appropriate event. Again, navigation to the appropriate event is performed by using input devices such as mechanical and/or touch sensitive and/or voice control parts. Next, (Step 7) the content of the display screen is automatically changed to prompt the user to apply a sample of blood on an application area of an appropriate test element. After a few moments, the concentration of blood glucose in a sample of blood is displayed on the display screen (Step 8). In addition, and as shown in FIG. 9c, the meal event category is displayed together with the optionally activated disease management compliance window thus forming a comprehensive tool for aiding a patient in managing a disease of interest. Of course it is entirely relevant for the above steps to be performed without the disease management compliance window to be switched on, i.e. that meal category selection is displayed without the graphical tool being displayed such as displayed on FIG. 9d.

Whilst the present invention may have particular applicability to personal glucose diagnostic devices, it is should be noted that the present invention is also applicable to other types of analytes e.g. cholesterol, alcohol, lactate and the like, and to sensors such as immunoassay sensors, coagulation sensors and the like.

Various embodiments of the invention have been described above. The descriptions are intended to be illustrative, not limitative. Thus, it will be apparent to one skilled in the art that certain modifications may be made to the invention as described without departing from the scope of the claims set out below.

1. A monitoring device comprising
a processor (6),
a built-in sensor or a port (14) equipped with a disposable sensor,
characterized in that
the device is adapted for relating the stored measurement values of the sensor to a medically useful compliance range.

2. The monitoring device of claim 1, wherein the compliance range extends from a lower limit to an upper limit.

3. The monitoring device of claim 1, wherein the device relates the stored measurement values of the sensor to a medical useful compliance range by determining one or more mean values and/or one or more statistical representations of the measurement values.

4. The monitoring device of claim 1, wherein the monitoring device is adapted for evaluating the measurement values of the sensor in accordance with a predefined set of specific events.

5. The monitoring device of claim 4, wherein the predefined set of specific events comprises one or more of: pre- and/or post-prandial meal time events, activities, medical treatment events.

6. The monitoring device of claim 5, wherein the specific events comprise pre- and/or post-prandial mealtime events, further preferably one or more of: pre-breakfast, post-breakfast, pre-lunch, post-lunch, pre-dinner, post-dinner, and/or bed-time, wake-up-time.

7. The monitoring device of claim 1, further comprising event input means for relating a current measurement value to a specific event.

8. The monitoring device of claim 7, wherein a specific event comprises a predefined time frame within a 24 hour time period.

9. The monitoring device of claim 7, wherein a specific event is defined by a time frame and an event marker.

10. The monitoring device of claim 4, wherein the predefined set of specific events forms a compliance management profile stored in the device memory.

11. The monitoring device of claim 10, wherein the compliance management profile reoccurs every 24 hours.

12. The monitoring device of claim 10, wherein the compliance management profile extends over a 24 hour time period and comprises one day and one night.

13. The monitoring device of claim 10, providing multiple compliance management profiles each specific to calculated averages and/or statistical representation of all measurements of a predefined number of days, i.e. the last 7, 14, or 30 days.

14. The monitoring device of claim 1, wherein the device provides a self-learning mode adapted for generating settings for a compliance management profile.

15. The monitoring device of claim 1, further comprising a display to show the relationship between the statistically processed measurement values and the compliance range graphically or using an iconic representation.

16. The monitoring device of claim 15, wherein the display is adapted for showing a compliance window with a compliance range and a current measurement indicator.

17. The monitoring device of claim 15, wherein the display is adapted for showing a compliance window with a compliance range, a mean measurement indicator and a current measurement indicator.

18. The monitoring device of claim 15, wherein the display is adapted for showing a compliance window with a lower limit and an upper limit indicator, a mean value indicator, and a current measurement indicator.

19. The monitoring device of claim 1, wherein a mean value is determined as an arithmetic mean of former measurements that have respectively occurred at a time of day defined by a time frame related to a specific event.

20. The monitoring device of claim 1, wherein the monitoring device is adapted for determining a mean value by averaging former measurement values that lie within the time frame corresponding to a specific event.

21. The monitoring device of claim 1, the monitoring device is adapted for determining a mean value by averaging former measurement values that lie within the time frame corresponding to a specific event, wherein the measurement values have been acquired during a predefined number of days.

22. The monitoring device of claim 1, wherein the predefined sets of a plurality of measurement values represents a mean value of measurements in a predefined number of days, such as the last 7 days, 14 days, or 30 days.

23. The monitoring device of claim 23, wherein the mean value of measurement values of a predefined number of days is determined for a specific event.

24. The monitoring device of claim 1, wherein the compliance range is provided in form of a graphical or iconic indicator to provide a compliance window to be targeted by the user.

25. The monitoring device of claim 24, wherein the left-hand side of compliance window represents a lower limit of the compliance range.

26. The monitoring device of claim 24, wherein the right-hand side of the compliance window represents an upper limit of the target compliance range.

27. The monitoring device of claim 24, wherein the center line of the compliance window represents the target value according to medically recommended analyte concentration.

28. The monitoring device of claim 1, having a compliance window for displaying on a display screen; the graphical or iconic compliance window comprising: a geometric shape suitable for a segmented liquid crystal display representing a scale for a deviation of the measurement values from the predetermined compliance range.

29. The monitoring device of claim 1, further comprising at least one mean value indicator representing a mean value of a plurality of measurement values acquired during a time frame related to a specific event.

30. The monitoring device of claim 1, further comprising an actual value indicator representing a current measurement value in relation to the at least one mean value indicator.

31. The monitoring device of claim 1, the monitoring device being adapted for determining, for a current measurement value, an updated mean value over a predetermined number of past days.

32. The monitoring device of claim 1, further comprising input means adapted for providing a scrolling function for scrolling through former measurement values and former mean values, both the former measurement values and the former mean values for a certain meal time event being displayed in the compliance window.

33. The monitoring device of claim 32, wherein by scrolling through former measurement values and former mean values, the trend of the movement of the mean values is visualized.

34. The monitoring device of claim 1, further comprising comparison means adapted for at least one of:
comparing a target value with an updated mean value,
comparing a target value with a current measurement value;
comparing a current measurement value with an updated mean value.

35. The monitoring device of claim 1, the monitoring device being equipped with an interactive navigational area having a selection window (80) located in its center, wherein one of the plurality of first level menu items is disposed while selected by the user.

36. The monitoring device of claim 35, wherein the interactive navigational area is provided on a display screen (38) for displaying a menu comprising a plurality of first level menu items in a sequential arrangement and being configured such that a selection of one of the plurality of first level menu items causes a plurality of second level menu items associated with one first level menu item to replace the first level menu items in the sequential arrangement.

37. The monitoring device of claim 36, wherein at least two of the plurality of first level menu items are disposed adjacent to the selection window during user operation.

38. The monitoring device of claim 35, wherein the interactive navigational area and the plurality of first level menu items include a text or an icon to represent a user operation.

39. The monitoring device of claim 35, wherein the plurality of first level menu items or second level menu items are populated according to a default configuration.

40. The monitoring device of claim 35, wherein the plurality of first level menu items or second level menu items are populated according to a user preference.

41. The monitoring device of claim 1, wherein the monitoring device is one of: a personal diagnostic device for disposable test elements, a personal battery powered diagnostic handheld device, a portable monitoring device.

42. The monitoring device of claim 1, further comprising an analog input device handling the user interaction and providing at least one selection button and at least one confirmation button.

43. The monitoring device of claim 42, wherein the analog input device is detecting the selection of a manual button, and/or a manual switch.

44. The monitoring device of claim 42, wherein the analog input device is adapted for detecting the change of capacitance and/or impedance and/or resistance on specially dedicated areas of the device housing to provide for user interactions.

45. The monitoring device of claim 28, wherein the compliance window and an interactive navigational area are provided on the same area of the display screen (38).

46. The monitoring device of claim 45, wherein the display screen (38) is used further to provide legible compliance information (38a).

47. The monitoring device of claim 45, wherein manipulation of compliance window and interactive navigational area is effected by operating at least one selection button and/or at least one confirmation button.

48. The monitoring device of claim 1, wherein the monitoring device is equipped and connected with an adaptor by means of a communication bus.

49. The monitoring device of claim 48, wherein the communication bus is a universal serial bus (USB).

50. The monitoring device of claim 36, wherein the display screen comprises at least one of: an organic light emitting diode, a dot matrix display screen, a segmented display screen, a hybrid dot matrix-segmented display screen.

51. The monitoring device of claim 41, wherein the personal diagnostic device is one or more of: a glucose diagnostic device, a coagulation diagnostic device, an immunoassay diagnostic device.

52. A method of using a monitoring device according to claim 1 comprising:
inserting a test element into the monitoring device;
activating the monitoring device automatically;
prompting meal time category automatically;
confirming meal time category by user;
prompting of user to apply blood sample of blood onto appropriate area of test element;
displaying measurement result with meal time indicator.

53. The method of using a monitoring device according to claim 52, wherein the user is informed by means of alphanumerical, graphical, or iconic illustration about his compliance stage and/or health condition.

54. The method of claim 52, further comprising
prompting pre or post meal time automatically;
confirming pre or post meal time by user.

55. A method according to claim 52, in which the automatic prompting of the meal time categories prompted to the user comprise one or more of: “Breakfast”, “Lunch”, “Dinner”, “Night”.
